(19) United States
(12) Patent Application Publication
(10) Pub. No.: US 2006/0058802 A1
(43) Pub. Date: Mar. 16, 2006

(54) Fixation implant for a bone graft within a joint for the purpose of ensuring arthrodesis of the joint

(76) Inventor: Hakon Kofoed, Charlottenlund (DK)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/220,144

(22) Filed: Sep. 6, 2005

(30) Foreign Application Priority Data
Sep. 6, 2004 (FR) FR-04 09427

(51) Int. Cl. A61F 2/30

(52) U.S. Cl. 606/75

(57) ABSTRACT

A fixation implant for a bone graft within a joint for the purpose of ensuring arthrodesis of the joint, and a surgical method for emplacement of the described fixation implant. The fixation implant for a bone graft is arranged between the bones located on both sides of an articular space, for the purpose of ensuring arthrodesis of a joint. The fixation implant (7) includes: at least two elements for anchoring (8) into the bones, connected to each other by at least one connection element (10) extending to the outside of the joint; and an immobilization means (11) for the bone graft, arranged between the anchoring elements (8) and connected to the connection element (10) in such a way as to ensure, in cooperation with the anchoring elements (8), blocking of the bone graft with respect to the bones of the joint and vice-versa.

### CROSS-REFERENCE TO RELATED APPLICATION

This Application claims priority to copending French Patent Application entitled, “Fixation Implant For a Bone Graft Within a Joint for the Purpose of Ensuring Arthrodesis of the Joint,” having Application No. FR-04 09427, filed on Sep. 6, 2004, which is entirely incorporated herein by reference.

### BACKGROUND

1. Field of the Invention

This invention relates to the general technical field of surgical fixation implants, and in particular to the sector of fixation implants designed to be used to achieve arthrodesis.

This invention relates more particularly to a fixation implant for a bone graft arranged between the bones located on both sides of an articular space, for the purpose of ensuring arthrodesis of a joint.

The fixation implant according to the invention is specifically designed to achieve arthrodesis of a joint, in particular, but not exclusively, the ankle joint.

This invention also relates to a surgical method for emplacement of the described fixation implant.

2. Discussion of the Related Art

Arthrodesis is a surgical intervention intended to almost completely suppress the mobility of a joint by causing “bone fusion.” Such a surgical intervention may prove to be necessary in the case in which the patient is suffering from severe and final arthrosis, or when the cartilage of the bones forming the joint is very damaged.

In order to carry out such interventions, we know to resect the damaged cartilaginous surfaces of the joint in such a way as to bring in contact, by compression, the facing bone surfaces of the bones forming the joint, thus making possible osteosynthesis.

However, such a method can lead to a shortening of the limb involved, which is quite obviously undesirable, not only from the aesthetic point of view, but also on account of the complications (excessive claudication, for example) which this may lead to.

In order to mitigate these disadvantages, we know to contrive a housing on both sides of the articular space by cutting out fragments of bone at the opposite ends of the bones forming the joint. This housing, preferably cylindrical, is then filled up with a bone graft, such as an approximately cylindrical core sample of bone taken from the same patient, for example from the iliac crest.

This manipulation thus makes it possible to dispose of the degraded cartilaginous surfaces and to replace the damaged bone ends with a healthy bone graft without shortening the limb in question. The bone graft is then fixed relative to the bones forming the joint with the aim of enabling osteosynthesis between the bone graft on the one hand and the bones on the other hand, thus ensuring arthrodesis of the joint.

Various fixation implants can be used for the purpose of ensuring fixation of the bone graft and arthrodesis of the joint. Thus, in the case of a joint between a first and a second bone, a Steinman pin is routinely used. The Steinman pin presents itself in the form of an elongated pin of length sufficient to successively cross the first bone of the joint, the bone graft and the second bone of the joint. Such fixation implants, while they make it possible to obtain significant results as far as joint immobilization is concerned, nevertheless suffer from non-negligible disadvantages.

In the first place, these fixation implants require, for their emplacement, an additional incision added to the incision already made for the purpose of putting in the housing designed for receiving the bone graft. This additional incision has the effect of significantly increasing the risk of infection and operatory or post-operatory complications.

In addition, Steinman pin fixation implants must generally be arranged in such a way as to extend obliquely or perpendicularly with respect to the articular space for the purpose of ensuring an effective maintenance of the joint. For certain joints, such as the ankle joint, such an orientation of the implant is undesirable because there is a risk that during patient management, and in particular during walking, the implant might cross the plantar cortex of the calcaneum and project outside the plantar facies.

Moreover, if the fixation implant is poorly positioned, it can also lead to damage of the soft tissues of the plantar facies. Now, fixation implants such as Steinman pins can prove to be difficult to position, in particular when the joints involve bones of small dimensions. Thus, in the case of poor orientation of the implant, it can happen that the bone graft is not maintained, the implant thus set up being then virtually ineffective.

### SUMMARY

One embodiment of a fixation implant for a bone graft arranged between bones located on both sides of an articular space for the purpose of ensuring arthrodesis of a joint includes at least two anchoring elements designed to be introduced into the bones, and equipped with a proximal end and a distal end, the distal end being adapted to be introduced into the bones. The anchoring elements are connected to each other by at least one connection element extending outside the joint. The implant includes an immobilization means for the bone graft arranged between the anchoring elements and connected to the connection element in such a way as to ensure, in cooperation with the anchoring elements, blocking of the bone graft with respect to the bones of the joint and vice-versa. The immobilization means is formed from a plate.

The objects assigned to the invention aim at remedying the various disadvantages previously enumerated and at proposing a novel fixation implant for a bone graft arranged between the bones located on both sides of an articular space, for the purpose of ensuring arthrodesis of a joint, which makes it possible to ensure, in a simple way, particularly effective and stable maintenance of the joint without risking damage to the cortex on the one hand, and the soft tissues in the neighbourhood of the bones forming the joint on the other hand.

Another object of the invention aims at proposing a novel fixation implant particularly suitable for immobilising a fragmented and/or fissured graft.

Another object of the invention aims at proposing a novel fixation implant adapted for effectively withstanding the mechanical stresses exerted on the joint, in particular during walking in the case of the ankle joint.

Another object of the invention aims at proposing a novel fixation implant that does not require an additional incision for its emplacement.

Another object of the invention aims at proposing a novel fixation implant that is less intrusive than known implants.

Another object of the invention aims at proposing a novel fixation implant presenting a structure and a shape adapted to the anatomy of the joint.

Another object of the invention aims at proposing a novel fixation implant that makes possible a solid and comfortable maintenance of the joint.

Another object of the invention aims at proposing a novel fixation implant whose manipulation is made easy and which makes possible reduction of operatory errors.

Other special features and advantages of the invention will appear in greater detail upon reading of the description which follows, and by means of the appended drawings provided in a purely illustrative and non-restrictive way.

### BRIEF DESCRIPTION OF THE DRAWINGS

FIG. 1 depicts, in a perspective view, an ankle joint with a bone graft arranged between the bones located on both sides of an articular space.

FIG. 2 depicts, in a perspective view, a fixation implant according to the invention in its functional position for maintenance of the joint.

FIG. 3 depicts, in a perspective view, a first embodiment of a fixation implant according to the invention.

FIG. 4 depicts, in a side view, another embodiment of a fixation implant according to the invention.

FIG. 5 depicts, in a frontal view, a fixation implant according to the invention in its functional position for compression of the bone graft and maintenance of the joint.

FIG. 6 depicts, in a cross-sectional view along line A-A depicted in FIG. 5, the fixation implant depicted in FIG. 5.

FIG. 7 depicts, in a perspective view, an ankle joint and a bone graft arranged between the bones located on both sides of the articular space, the bone graft being formed by two fragments separated by the articular space.

FIG. 8 depicts, in a perspective view, a fixation implant according to the invention put in place within the joint depicted in FIG. 7.

FIG. 9 depicts, in a side view in perspective, an embodiment of the fixation implant according to the invention, equipped with a compression unit.

FIG. 10 depicts, in a side view in perspective, an improved variant of realisation of the fixation implant equipped with a compression unit according to the invention.

FIGS. 11 to 13 depict, in side views in perspective, various embodiments of a compression unit according to the invention.

FIG. 14 depicts, in a schematic view, an implant according to another embodiment of the invention.

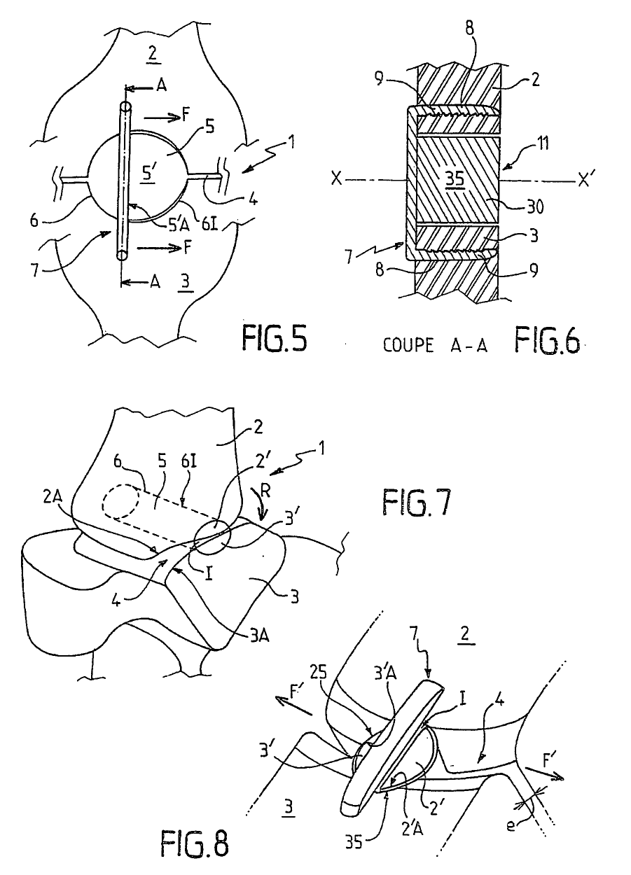

FIGS. 1 and 7 also depict two surgical methods that make it possible to achieve arthrodesis of the ankle joint.

### DETAILED DESCRIPTION

Arthrodesis of a joint becomes necessary when the joint is in such a degraded condition that other less severe surgical interventions, such as for example those consisting in the placement of prostheses, prove to be ineffective. It becomes necessary, in this case, to completely immobilize the joint. This invention is depicted in the case of an ankle joint but could be applied to all types of joints in the human or animal body.

A damaged joint is characterized in particular by the condition of the cartilage of the bones delimiting the articular space. In the case of severe arthrosis, this cartilage is particularly worn out and can lead to pain or inflammation of the joint.

FIGS. 1 and 7 depict a joint 1 formed from at least two bones, namely a first bone 2, and a second bone 3 located on both sides of an articular space 4. However, joint 1 could quite obviously comprise a third bone, for example located between the first bone 2 and the second bone 3, and this without leaving the framework of the invention.

Several arthrodesis techniques are conceivable but the fixation implant according to the invention is more specifically designed to be used for the purpose of achieving arthrodesis by means of a bone graft 5 arranged between bones 2 and 3 located on both sides of the articular space 4. A first method that is known thus consists in putting in a housing 6, for example cylindrical, in the ends 2A and 3A of bones 2 and 3 delimiting the articular space 4.

According to this first known method, the bone fragments contained in the housing 6 are extracted and a bone core sample is taken from another part of the patient's body, for example from the iliac crest, with the purpose of introducing it within the housing 6 once the bone fragments are withdrawn. This core sample constitutes, after its emplacement within the housing 6, a bone graft 5, approximately solid, i.e. neither hollow nor split, suitable to be fixed relative to the bones 2 and 3 for the purpose of ensuring osteosynthesis between the bone graft 5 and bones 2 and 3. Specifically, osteosynthesis is achieved between the bleeding outer bone surface 5A of the bone graft 5 and the surface of section 6A, also bony, of bones 2 and 3. For the purpose of the invention, the bleeding outer bone surface 5A of the bone graft 5 corresponds to the surface according to which the bone graft 5 was cut out. In the case of a cylindrical core sample, the bleeding outer bone surface 5A therefore corresponds to the lateral surface of the core sample. The surface of section 6A corresponds approximately, for the purpose of the invention, to the inner wall 61 of the housing 6.

A variant of this method was invented by the applicant. Like the preceding method, the method according to the applicant's invention consists in putting in a housing 6, preferably cylindrical, on both sides of the articular space 4. On the other hand, the contents of housing 6 are not hollowed out, i.e. there is no withdrawal of bone fragments 2′ and 3′ cut out, respectively, from the ends of bones 2 and 3 located on both sides of the articular space 4. Bone fragments 2′ and 3′, juxtaposed within the housing 6 and separated by an interstice I derived for example from the articular space 4 then constitute the bone graft 5. The worn-out cartilaginous surfaces of the joint are thus located on both sides of the interstice I separating the bone fragments 2′ and 3′ and therefore at the centre of the bone graft 5.

The applicant's method consists then in displacing the bone graft 5, formed from bone fragments 2′ and 3′, within its housing 6, by making it turn on itself for example by a quarter turn in the direction of rotation R indicated in FIG. 7. In this way, the bleeding outer bone surface 2′A of the first bone fragment 2′ is put facing the surface of the section 3S, also bony, of the second bone 3. In the same way, the bleeding outer bone surface 3′A of the second bone fragment 3′ is put facing the surface of section 2S of the first bone 2. This thus makes possible osteosynthesis of bone fragments 2′ and 3′ with, respectively, bones 3 and 2 and more generally osteosynthesis of the bone graft 5 with bones 2 and 3.

For the purpose of the invention, the expression “bone graft” therefore refers either to a bone transplant, i.e. a piece of bone, preferably of one piece, taken from a part of the body located at a distance from the joint 1 and introduced within the housing 6, or to an assembly of bone fragments 2′ and 3′ cut out within the joint 1 during construction of the housing 6, and which has been caused to undergo a displacement, such as a rotation R within the housing 6. The bone graft 5 therefore constitutes a unitary totality, formed either from a one-piece bone fragment, or by a plurality of bone fragments juxtaposed within the housing 6.

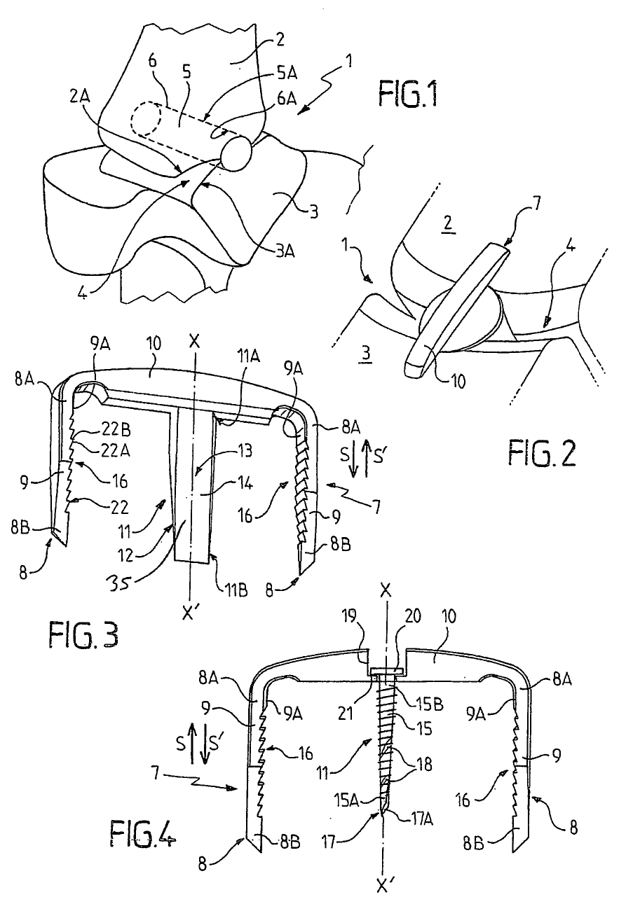

FIGS. 3 to 6 depict several variants of realisation of a fixation implant 7, designed in particular to be used to fix the bone graft 5, in one piece, depicted in FIG. 1.

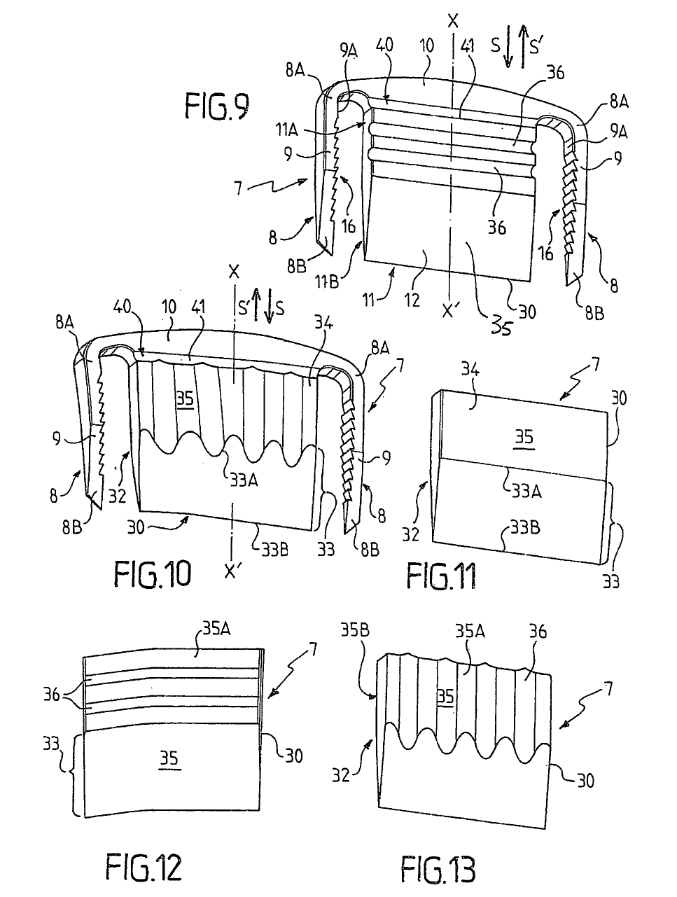

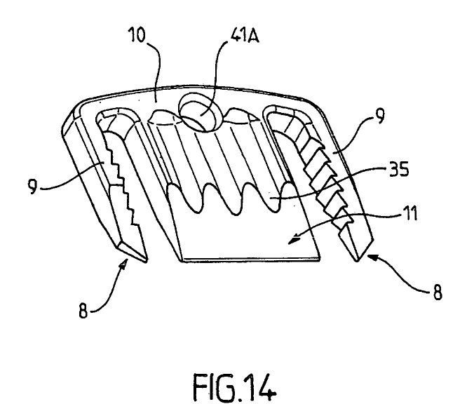

FIGS. 9 to 14 depict other variants of realisation of the fixation implant 7, designed in particular to be used to ensure fixation of the bone graft 5 formed by the bone fragments 2′ and 3′ depicted in FIG. 7.

According to the invention, the fixation implant 7 comprises at least 2 anchoring elements 8 designed to be introduced in bones 2 and 3. Preferentially, the fixation implant 7 comprises as many anchoring elements 8 as the joint comprises bones 2 and 3. Thus, if the joint 1 is formed from two bones 2 and 3, the fixation implant will preferably comprise two anchoring elements 8, as is shown in FIGS. 3, 4 and 9. It is however quite obviously conceivable to equip the fixation implant with several anchoring elements for the same bone, and this without leaving the framework of the invention.

Each anchoring element 8 extends preferentially between a proximal end 8A and a distal end 8B. The distal end 8B is in addition adapted to be introduced into bones 2 and 3 and is to that end preferentially tapered, or pointed. By means of this technical measure, each anchoring element 8 presents a self-perforating character, making possible the penetration of the anchoring element 8 into the bone.

The anchoring elements 8 are to best advantage connected to each other through at least one connection element 10. As depicted in FIGS. 2 and 8, the connection element 10 extends outside of the joint 1 and overlaps the articular space 4, thus forming an essentially rigid connection bridge between the anchoring elements 8 and therefore between bones 2 and 3. This connection bridge 10 thus confers on the fixation implant 7 its structural rigidity, which makes it possible for it to better withstand the various mechanical stresses to which the joint 1 is subjected. The connection element 10 being arranged, in its functional position, between the joint 1 and the soft tissues (not shown), it preferably presents rounded edges which confer upon it an essentially non-invasive character with respect to the surrounding soft tissue.

Preferentially, the anchoring elements 8 are formed from anchoring arms 9 whose distal ends 8B are substantially tapered in such a way as to facilitate their penetration into the bone tissues.

To best advantage, the anchoring elements 8, in particular the anchoring arms 9, extend essentially parallel to the articular space 4, and are essentially perpendicular to the connection element 10. The fixation implant 7 then presents to best advantage a U shape, the arms of the U being formed from the anchoring arms 9, and the base of the U being formed from the connection element 10.

It is, however, quite obviously conceivable to construct a fixation implant whose anchoring arms 9 extend obliquely and not perpendicularly with respect to the connection element 10 and in such a way as to come close to each other. The anchoring arms 9 can thus to best advantage be constructed from a material with shape memory so as to shrink, and to come close to each other once introduced into bones 2 and 3, in order to ensure effective compression of bones 2 and 3 against each other.

According to the invention, the fixation implant 7 also comprises a means for immobilization 11 of the bone graft 5, arranged between the anchoring elements 8 and connected to the connection element 10 in such a way as to ensure, in cooperation with the anchoring elements 8, blocking of the bone graft 5 with respect to bones 2 and 3 of the joint 1 and conversely.

The expression “in cooperation” refers to the fact that the immobilization means 11 acts together with the anchoring elements 8 to immobilize the bone graft 5 with respect, on the one hand, to the anchoring elements 8 and, on the other hand, to bones 2 and 3 of the joint 1. To best advantage, the immobilization means 11 extends longitudinally, in a longitudinal direction X-X′ approximately parallel to the articular space 4, and this contrary to the devices of prior art, such as Steinman pins, which extend in an essentially perpendicular or oblique fashion with respect to the articular space.

The immobilization means 11 presents, of course, a shape and dimensions adapted for ensuring stable and reliable immobilization of the graft. In that, the immobilization means is not simply a means for indexing the implant in position relative to the graft, but rather a means for mechanical embedding of the implant in the graft.

The immobilization means 11 is also distinct from and exogenous to the graft and adapted for cooperation with the latter. The immobilization means 11 therefore does not directly, of itself, form, for the purpose of the invention, a substrate for growth or bone or tissue regeneration, but rather a unit interacting mechanically with the graft to block the latter.

The immobilization means 11 thus to best advantage extends, in the longitudinal direction X-X′, between a proximal part 11A connected to the connection element 10 and a distal part 11B arranged on the opposite side from the proximal part 11A. The immobilization means 11 is thus arranged perpendicularly to the connection element 10.

Preferentially, the anchoring arms 9 extend longitudinally in an essentially parallel fashion in the longitudinal extension direction X-X′ of the immobilization means 11. In addition, the anchoring arms 9 present to best advantage, along their length, a variable thickness which decreases between their proximal end 8A and their distal end 8B in such a way as to facilitate their penetration into bones 2 and 3.

As depicted in FIG. 3, the anchoring arms 9 preferentially have the same length as the immobilization means 11. It is, however, quite obviously conceivable to construct anchoring arms 9 of different lengths. Thus, it is conceivable to construct a fixation implant 7 equipped with anchoring arms 9 appreciably longer than the immobilization means 11 (FIG. 4).

To best advantage, the immobilization means 11 comprises a unit for introduction 12 within the bone graft 5. The introduction unit 12 is thus adapted to penetrate either inside a one-piece bone graft 5, formed from a single bone fragment (FIG. 2), or into the interstice I separating bone fragments 2′ and 3′ within the bone graft 5 (FIGS. 7 and 8). To this end, the distal part 11B of the immobilization means 11, which forms the introduction unit 12, is preferably shaped to a point or tapered.

In order to ensure effective maintenance of the bone graft 5 and to make possible osteosynthesis with bones 2 and 3, the immobilization means 11 comprises to best advantage means for rotation blocking 13 adapted to prevent rotation of the bone graft 5 around the fixation implant 7, and vice-versa.

As depicted in FIG. 3, the rotation blocking means 13 are to best advantage formed from at least one flat part 14, arranged along the immobilization means 11. The immobilization means 11 can thus be presented in the shape of a point comprising at least one essentially flat outer surface forming flat part 14.

In accordance with the invention, as depicted in FIGS. 3, 6 and 9 to 14, the immobilization means 11 is formed from at least one plate 35, i.e. from a two-dimensional element. By “two-dimensional element, ” an element of flattened shape is designated here, whose thickness is slight compared to its length and width. In other words, such an immobilization means 11 presents a blade shape and extends principally in two directions of space, and not essentially in a single direction of space, like the screw 15 described in more detail below.

Plate 35 can, for the purpose of the invention, present a relatively spread-out shape, as in the variant of FIGS. 9 to 14, or slender, as in the variant of FIG. 3.

Implementation of an immobilization means 11 formed from a plate 35 makes possible excellent maintenance of the bone graft 5, and in particular allows use of a graft that is fissured, split or even broken into several fragments. In this case, implementation of an immobilization means 11 in plate shape makes it possible, in fact, to ensure a relative blocking of the fragments that is sufficiently robust and stable to withstand the loads to which the joint may be subjected (in particular when the latter is an ankle joint, on which the weight of the patient's body is exerted).

The plate 35 can have a thickness that is essentially uniformly constant. In the case in which the graft is formed from several fragments, the dimensions of the plate, and in particular its thickness, will preferably be chosen as a function of the free space between the fragments in such a way that the plate occupies a volume sufficient to block the fragments.

Plate 35 can, however, possibly be made up of several sections, each presenting a thickness that is essentially constant and different from the thickness of the other sections. In this case, coupling between each section can be sharp, and be presented for example in the shape of a shoulder or a “step.”

It is also conceivable, in a preferential variant of realisation depicted in FIGS. 3 and 9 to 13, and described in more detail below, that the plate 35 present a “wedge-like,” tapered shape, i.e. whose thickness increases progressively, in the longitudinal direction, over at least a part of the plate, from its distal end 11B towards its proximal end 11A.

In this case, described in more detail in what follows, the immobilization means forms (or is formed) from a compression unit 30.

According to a variant of realisation of the invention depicted in FIG. 4, the immobilization means 11 may, however, be formed from a screw 15, preferably self-drilling and self-threading, and equipped to this end with preparation means 17 formed from at least one tooth 17A extending essentially axially in the longitudinal direction X-X′.

To best advantage, the screw 15 also comprises grooves 18 arranged along its length in such a way as to make possible progressive evacuation of excess bone matter during its screwing inside the bone graft 5.

According to a first embodiment of the invention, depicted in FIG. 4, the immobilization means 11, for example the screw 15, is mounted in a removable way on the connection element 10. The latter is to this end to best advantage equipped with a through-hole 19, preferentially fitted approximately in the centre of the connection element 10 and adapted to receive the immobilization means 11. Thus, the screw 15 can to best advantage comprise, towards its proximal end 15B, a head 20 designed to support a shoulder 21, forming an abutment, fitted within through-hole 19.

According to another variant of realisation of the invention depicted in FIGS. 3, 9 and 10, the immobilization means 11 is to best advantage permanently united with the connection element 10 and for example made in one piece with the latter, thus forming a one-piece totality.

Preferentially, and as depicted in FIGS. 3 and 4, the anchoring means 8 are formed from two lateral and spaced-out anchoring arms 9, arranged oppositely on both sides of the connection element 10, parallel with respect to each other. The anchoring arms 9 are preferably made in one piece with the connection element 10 but can quite obviously be formed from distinct parts of the connection element 10, and united with the latter, for example by means of fixation screws (variant not shown). In a particularly advantageous way, the anchoring arms 9 are preferentially identical and symmetrically arranged on both sides of the immobilization means 11.

In addition, the immobilization means 11 is to best advantage formed from a central arm approximately parallel to the lateral anchoring arms 9, in such a way as to extend perpendicularly with respect to the connection element 10.

In an even more preferential way, the anchoring arms 9 and the immobilization means 11 are made of one piece, thus forming a one-piece fixation implant 7. Such a fixation implant withstands particularly well the mechanical stresses to which the joint is subjected.

To best advantage, and as shown in FIGS. 3, 4, 9 and 10, the anchoring arms 9 are equipped with reverse-lock means 16 specifically designed to prevent displacement of the fixation implant 7 in a direction S′ opposite to its direction of introduction S into the bone graft 5. To best advantage, the reverse-lock means 16 are preferentially formed from at least one protuberance 22 protruding from the outer surface of the anchoring arms 9. In an even more preferential way, the reverse-lock means 16 are formed from a plurality of protuberances 22 arranged along the anchoring arms 9, in the longitudinal direction X-X′.

The anchoring arms 9 comprise to best advantage an inner surface 9A, located essentially facing the immobilization means 11, on which the reverse-lock means 16 are arranged. The inner surface 9A thus presents a notched appearance, each protuberance 22 forming a notch and presenting an inclined surface 22A designed to facilitate the introduction of the anchoring arms 9 into the bone tissue, and a horizontal surface 22B, approximately perpendicular to longitudinal direction X-X′ and in the direction S of introduction of the fixation implant 7 so as to prevent disengagement of said fixation implant 7 once the latter is put in place within the joint 1.

According to a particularly advantageous characteristic of the invention, the immobilization means 11 is formed from a compression unit 30 adapted to support the bone graft 5 and to exert on the latter a pressure sufficient so that the bone graft 5 supports, at least partially, bones 2 and 3 of the joint 1 in such a way as to promote osteosynthesis between the bone graft 5 and bones 2 and 3.

In the case of the configuration depicted in FIG. 5, in which the bone graft is formed from a single fragment of bone 5′, preferably of one piece, arranged within the housing 6, the compression unit 30 is adapted to support at least part of the outer surface 5′A of bone fragment 5′ in such a way as to compress the latter in a direction of compression F against the inner wall 61 of the housing 6.

To this end, as was set forth in the preceding, the compression unit 30 is preferentially formed from a plate 35, one of whose surfaces comes in contact with the outer surface 5′A of bone fragment 5′. Bone fragment 5′ can to best advantage be presented in the shape of a hemicylindrical block not occupying all of the housing 6, presenting an approximately flat portion of the outer surface 5′A which the plate 35 is capable of supporting.

According to the method depicted in FIGS. 7 and 8, the bone graft 5 comprises at least two bone fragments 2′ and 3′, separated by the interstice I. The compression unit 30 is then adapted to be introduced within the interstice I, with a low clearance, and to exert external centrifugal or radial compression, following arrows F′, on bone fragments 2′ and 3′, for the purpose of pushing them back against the inner wall 61 of the housing 6 and thus ensuring the expansion of the bone graft 5 and its blocking within the housing 6, and more generally within the joint 1.

According to a particularly advantageous characteristic of the invention, the compression unit 30 is equipped with progressive spreading means 32, adapted to ensure, as the penetration of the compression unit 30 into interstice I progresses, progressive compression of bone fragments 2′ and 3′. As depicted in FIG. 10, the spreading means 32 are to best advantage formed from a section 33 of the compression unit 30 whose thickness is variable. Thus section 33 extends, in the direction of introduction S of the compression unit 30, between a proximal limit 33A, located on the side of the connection element 10, and a distal limit 33B opposite. The thickness of section 33 increases to best advantage, for example continuously, between distal limit 33B and proximal limit 33A in such a way as to ensure progressive spreading of bone fragments 2′ and 3′.

It is, however, conceivable that the thickness of section 33 could be approximately constant, or could vary by sharp levels, without on this account leaving the framework of the invention.

In a particularly advantageous way, the compression unit 30 comprises a tapered distal part, designed to facilitate its introduction into the bone graft 5, and to best advantage formed from the spreading means 32. The compression unit 30 in addition comprises a proximal part 34 essentially thicker than its distal part which corresponds to section 33. In an even more preferential way, proximal part 34 presents a thickness approximately equal to the width of the interstice I, itself approximately identical to the width of the articular space 4, so as to avoid the phenomenon of the shortening of the limb of the patient comprising the joint 1.

To best advantage, the compression unit 30 is formed from a plate 35, approximately prismatic and flattened, forming a wedge. Plate 35 can to best advantage be formed from a material with shape memory, and be designed to expand after its introduction within the interstice I for the purpose of ensuring compression and progressive spreading of bone fragments 2′ and 3′. The plate 35 preferably comprises two surfaces 35A and 35B, at least one of said surfaces 35A comprising grooves 36.

According to a first variant of realisation depicted in FIG. 10, grooves 36 can to best advantage extend in a direction approximately parallel to longitudinal extension direction X-X′ of the compression unit 30. Such a configuration in particular makes it possible to appreciably improve the effectiveness of compression.

According to another variant depicted in FIG. 9, the grooves 36 preferably extend in a direction approximately perpendicular to longitudinal extension direction X-X′ of the compression unit 30. According to this configuration, the grooves 36 to best advantage form reverse-lock means opposing extraction of the implant once the latter is introduced within the bone graft 5.

According to a preferential variant depicted in particular in FIG. 10, the immobilization means 11 or the compression unit 30, formed from the plate 35, extends in a principal extension plane P and the anchoring arms 9 are to best advantage located in this same principal extension plane P.

According to an advantageous characteristic of the invention, the fixation implant 7 comprises gripping means 40 to best advantage formed from at least one groove and preferably two grooves 41 arranged on both sides of the fixation implant 7, preferentially between the connection element 10 and the immobilization means 11.

Alternatively, as depicted in FIG. 14, gripping means 40 are formed from an orifice 41A fitted through the thickness of the implant, preferably in the neighbourhood of the junction between the immobilization means and the connection element. This orifice 41A makes possible the introduction of an extraction instrument in the shape of a rod, in particular making it possible to exert a lever arm on the implant in order to remove it from the bones if necessary.

In a particularly advantageous embodiment, and which moreover constitutes an invention in its own right, the compression unit 30 is independent of the anchoring elements 8, i.e. it is not connected to the latter by means of the connection element 10.

The fixation implant 7 is then formed exclusively from the compression unit 30, and does not comprise the anchoring element 8.

As depicted in FIGS. 11 to 13, the compression unit 30 comprises all characteristics previously described but is to best advantage designed to be introduced within the bone graft 5 independently of the anchoring elements 8. It is then possible to use other fixation means, for example fixation screws, in order to unite bone fragment 5′ or each of fragments 2′ and 3′ to bones 2 and 3, independently of the compression unit 30.

The compression unit 30 is then, according to the case, specifically adapted to support bone fragments 5′ or 2′ and 3′ and to exert a sufficient pressure on the latter to push them back against the inner wall 61 of the housing 6 and to ensure blocking of the bone graft 5 within joint 1.

Whatever their embodiment, the fixation implant 7, the anchoring elements 8, the immobilization means 11 or the compression unit 30 are to best advantage made of a bio-resorbable material, which makes it possible to avoid a new surgical intervention for the purpose of withdrawing them.

The surgical method for emplacement of the fixation implant 7 will now be described with reference to FIGS. 1 to 13.

The surgical method according to the invention includes, subsequently to the step of emplacement or positioning of the bone graft 5 within its housing 6, a step for fixation of the bone graft 5 by means of the fixation implant 7 previously described.

This fixation step comprises at first a step a) of immobilization relative to bones 2 and 3 forming the joint. This step a) is carried out by means of one or more impacts, performed for example by means of an appropriate impactor, on the implant, and for example either on the connection element 10, or on the anchoring elements 8, in such a way as to cause the latter to penetrate into each of bones 2 and 3 of the joint 1. Bones 2 and 3 are then united with each other through the connection element 10.

The surgical method also includes a step b) for blocking of the bone graft 5 with respect to bones 2 and 3 forming the joint. This step b) can be carried out simultaneously with step a) or subsequently to step a), in particular when the immobilization means 11 is not of one piece with the anchoring elements 8. In the case in which the immobilization means 11 is formed from an independent part, it is possible to introduce the latter within the bone graft 5 in particular by means of one or more impacts carried out on the proximal part 11A of the immobilization means 11. This way of proceeding can prove to be judicious for example in the case in which the immobilization means 11 is formed from a prismatic plate 35 forming a wedge. On the other hand, if the immobilization means 11 is formed from a screw 15, step b) will preferentially be carried out by introducing the screw 15 within through-hole 19 and then screwing the latter inside the bone graft 5, for example within one-piece bone fragment 5′.

In the case in which the immobilization means 11 is of one piece with the anchoring elements 8, the immobilization means 11 is introduced within bone graft 5 at approximately the same time as the anchoring elements 8 penetrate inside bones 2 and 3 under the action of the impactor.

The surgical method according to the invention additionally comprises to best advantage a step c) for compression of the bone graft 5 within the housing 6 arranged on both sides of the articular space 4.

To best advantage, steps a), b) and c) are carried out simultaneously.

In particular, the bone graft 5 being formed from at least two bone fragments 2′ and 3′, step c) involves the exertion of an external radial or centrifugal compression, following arrows F′, on bone fragments 2′ and 3′ for the purpose of pushing them back towards the inner wall 61 of the housing 6, thus ensuring their blocking as well as that of the bone graft 5, on the one hand within the housing 6 and on the other hand within the joint 1.

Such a step can be conducted independently of steps a) and b) described previously and then constitutes an invention in its own right. This step is to best advantage carried out by introducing the compression unit 30 within the interstice I between bone fragments 2′ and 3′, said compression unit 30 being or not being associated with the anchoring elements 8 in order to form the fixation implant 7.

The fixation implant 7 according to the invention therefore makes it possible to ensure, temporarily or permanently, effective maintenance of the joint 1 and the bone graft 5 within said joint 1, and therefore to facilitate osteosynthesis between the bone graft 5 and each of bones 2 and 3.

Another advantage of the fixation implant 7 according to the invention derives from its ease of emplacement, by means of a simple impactor, and without an additional incision being necessary for its implantation.

At least the following is claimed:

1. A fixation implant for a bone graft (5) arranged between bones (2, 3) located on both sides of an articular space (4) for the purpose of ensuring arthrodesis of a joint (1), said fixation implant (7) comprising:
at least two anchoring elements (8) designed to be introduced into the bones (2, 3), and equipped with a proximal end (8A) and a distal end (8B), said distal end (8B) being adapted to be introduced into the bones (2, 3), and said anchoring elements (8) being connected to each other by at least one connection element (10) extending outside the joint (1); and
an immobilization means (11) for the bone graft (5), arranged between the anchoring elements (8) and connected to the connection element (10) in such a way as to ensure, in cooperation with the anchoring elements (8), blocking of the bone graft (5) with respect to the bones (2, 3) of the joint (1) and vice-versa,
the immobilization means (11) being formed from a plate (35).

2. The implant of claim 1, wherein the immobilization means (11) comprises a unit for introduction (12) within the bone graft (5).

3. The implant of claim 2, wherein the immobilization means (11) extend, in a direction referred to as longitudinal (X-X′), between a proximal part (11A) connected to the connection element (10), and a tapered distal part (11B), forming the introduction unit.

4. The implant of claim 1, wherein the immobilization means (11) comprises rotation blocking means (13) adapted to prevent rotation of the bone graft (5).

5. The implant of claim 4, wherein the rotation blocking means (13) are formed from at least one flat part (14), arranged along the immobilization means (11).

6. The implant of claim 1, wherein the immobilization means (11) is mounted in a removable way on the connection element (10).

7. The implant of claim 6, further comprising a through-hole (19), fitted through the connection element (10), and adapted for receiving the immobilization means (11).

8. The implant of claim 1, wherein the immobilization means (11) is permanently united with the connection element (10).

9. The implant of claim 1, wherein the immobilization means (11) is formed from a compression unit (30), adapted to support the bone graft (5), and to exert on the latter a pressure sufficient so that the bone graft (5) supports, at least partially, the bones (2 and 3) of the joint (1) in such a way as to promote osteosynthesis between the bone graft (5) and said bones (2 and 3).

10. The implant of claim 9, wherein the bone graft (5) is formed from at least one bone fragment (5′), arranged within a housing (6) fitted in the ends of the bones (2 and 3) located on both sides of the articular space (4), said housing (6) being equipped with an inner wall (61), the compression unit (30) is adapted to support the outer surface (5′A) of bone fragment (5′) in such a way as to compress the latter against the inner wall (61) of the housing (6).

11. The implant of claim 10, wherein the bone graft comprises at least two bone fragments (2′ and 3′), separated by an interstice (I), said compression organ (30) is adapted to be introduced within the interstice (I) and to exert an external radial compression on bone fragments (2′ and 3′) for the purpose of pushing them back against the inner wall (61) of the housing (6) and thus ensuring the expansion of the bone graft (5) and its blocking within the joint (1).

12. The implant of claim 11, wherein the compression unit (30) is equipped with progressive spreading means (32) adapted to ensure, as its penetration into the interstice (I) progresses, progressive compression of the bone fragments.

13. The implant of claim 12, wherein the spreading means (32) are formed from a section (33) of the compression unit; extending, in the direction of introduction (S) of the compression unit (30), between a proximal limit (33A), located on the side of the connection element (10), and a distal limit (33B) opposite, the thickness of said section (33) increasing substantially between the distal limit (33B) and the proximal limit (33A).

14. The implant of claim 9, wherein the compression unit (30) comprises a tapered distal part, designed to facilitate its introduction into the bone graft (5).

15. The implant of claim 9, wherein the compression unit (30) comprises a proximal part (34) substantially thicker than the distal part.

16. The implant of claim 9, wherein the compression unit (30) is formed from an essentially prismatic and flattened plate (35).

17. The implant of claim 16, wherein said plate (35) comprises two principal surfaces (35A and 35B) that are essentially parallel, at least one of said surfaces (35A and 35B) comprising grooves (36).

18. The implant of claim 17, wherein the grooves (36) extend in a direction approximately parallel to the longitudinal extension direction (X-X′) of the compression unit (30).

19. The implant of claim 17, wherein the grooves (36) extend in a direction approximately perpendicular to the longitudinal extension direction (X-X′) of the compression unit (30).

20. The implant of claim 1, wherein the anchoring elements (8) are formed from anchoring arms (9), the distal ends (8B) of said anchoring arms (9) being substantially tapered so as to facilitate their penetration into the bone tissues.

21. The implant of claim 20, wherein the anchoring arms (9) extend longitudinally essentially parallel to the longitudinal extension direction (X-X′) of the immobilization means (11).

22. The implant of claim 20, wherein the immobilization means (11) extends in a principal extension plane (P), and in that the anchoring arms (9) are located essentially in said principal extension plane (P).

23. The implant of claim 20, wherein the anchoring arms (9) present, longitudinally, a variable thickness.

24. The implant of claim 20, wherein the anchoring arms (9) are approximately the same length as the immobilization means (11).

25. The implant of claim 20, wherein the anchoring arms (9) have different lengths.

26. The implant of claim 20, wherein the anchoring arms (9) are equipped with reverse-lock means (16), designed specifically to prevent displacement of the fixation implant (7) in a direction (S′) opposite to its direction of introduction (S) into the bone graft (5).

27. The implant of claim 26, wherein the reverse-lock means (16) are formed from at least one protuberance (22) projecting from the outer surface of the anchoring arms (9).

28. The implant of claim 27, wherein the reverse-lock means (16) are formed from a plurality of protuberances (22) arranged along the anchoring arms (9).

29. The implant of claim 26, wherein the anchoring arms (9) comprise an inner surface (9A), located approximately facing the immobilization means (11), the reverse-lock means (16) being arranged on said inner surface (9A).

30. The implant of claim 1, further comprising gripping means (40).

31. The implant of claim 30, wherein the gripping means (40) is formed from two grooves (41), arranged on both sides of the fixation implant (7).

32. The implant of claim 30, wherein the gripping means (40) is formed from an orifice (41A) fitted through the thickness of the implant, preferably in the neighbourhood of the junction between the immobilization means and the connection element.

33. The implant of claim 1, wherein the anchoring means (8) are formed from two spaced-out lateral anchoring arms (9), arranged opposite each other on both sides of the connection element (10), parallel with respect to each other, and in that the immobilization means (11) is formed from a central arm approximately parallel to the anchoring arms (9), said anchoring arms (9) and said central arm extending perpendicularly with respect to the connection element (10).

34. The implant of claim 1, wherein the connection element (10) is arranged so as to overlap the articular space (4).

35. A surgical method for emplacement of a fixation implant for a bone graft (5) arranged between bones (2 and 3) located on both sides of an articular space (4), for the purpose of ensuring arthrodesis of a joint (1), said method comprising
fixing a bone graft (5) by means of a fixation implant, the implant comprising:
at least two anchoring elements (8) designed to be introduced into the bones (2, 3), and equipped with a proximal end (8A) and a distal end (8B), said distal end (8B) being adapted to be introduced into the bones (2, 3), and said anchoring elements (8) being connected to each other by at least one connection element (10) extending outside the joint (1); and
an immobilization means (11) for the bone graft (5), arranged between the anchoring elements (8) and connected to the connection element (10) in such a way as to ensure, in cooperation with the anchoring elements (8), blocking of the bone graft (5) with respect to the bones (2, 3) of the joint (1) and vice-versa, the immobilization means (11) being formed from a plate (35).

36. The surgical method of claim 35 in which the step of fixing the bone graft comprises a step (a) for immobilization relative to the bones (2 and 3) forming the joint.

37. The surgical method of claim 36 in which step (a) includes impacting the implant to cause the anchoring elements 8 to penetrate into each of the bones (2 and 3).

38. The surgical method of claim 37, further comprising a step (b) for blocking of the bone graft (5) with respect to the bones (2 and 3) forming the joint.

39. The surgical method of claim 38 comprising a step (c) for compression of the bone graft (5).

40. The surgical method of claim 38, wherein steps (a) and (b) are carried out simultaneously.

41. The surgical method of claim 39, wherein steps (a), (b) and (c) are carried out simultaneously.
